(19) United States
(12) Patent Application Publication
(10) Pub. No.: US 2017/0312092 A1
(43) Pub. Date: Nov. 2, 2017

(54) FULLY EXPANDABLE INTERVERTEBRAL FUSION IMPLANT WITH TRAVELLER

(71) Applicant: FACET-LINK INC., Rockaway, NJ (US)

(72) Inventors: Helmut D. LINK, Hamburg (DE); Klaus DMUSCHEWSKY, Hamburg (DE)

(73) Assignee: FACET-LINK INC., Rockaway, NJ (US)

(21) Appl. No.: 15/522,672

(22) PCT Filed: Oct. 30, 2015
(86) PCT No.: PCT/EP2015/075256

§ 371 Date: Apr. 27, 2017

(30) Foreign Application Priority Data
Oct. 31, 2014 (EP) 14191310.3

(51) Int. Cl. A61F 2/44; A61F 2/46; A61F 2/30

(52) U.S. Cl. CPC A61F 2/447; A61F 2/4611; A61F 2/4684; A61F 2/4425; A61F 2002/30579; A61F 2002/30556; A61F 2002/30538; A61F 2002/30537; A61F 2002/30471; A61F 2002/304; A61F 2002/30176; A61F 2002/30156; A61F 2002/30601; A61F 2002/30617; A61F 2002/4627; A61F 2002/4623

(57) ABSTRACT

An intervertebral fusion implant for fusion of adjacent vertebrae, includes a main body having a first leg, a second leg, and an intermediate articulated joint, a pivoting blade is rotatably attached to the first leg, a guiding trough that includes two lateral side walls and two end-faces, one being stationary and one being movable and formed by a traveller movable along the guiding trough to vary its distance to the stationary end-face, the traveller being rotatably attached to the other end of the pivoting blade, wherein an attachment device is provided at the stationary end-face, and a second attachment device is provided at the traveller, which includes an aperture for passage of the holding instrument, wherein a lifting mechanism includes a lifting plate adjustable between retracted and raised states, the lifting plate being spaced from a top surface of the legs and forming a bearing for an adjacent vertebrae end-plate.

### CROSS-REFERENCE TO RELATED APPLICATIONS

This application is a National Phase application under 35 U.S.C. §371 of International Application No. PCT/EP2015/075256 filed Oct. 30, 2015, which claims priority benefit to European Patent Application No. 14191310.3 filed Oct. 31, 2014, the disclosures of which are herein incorporated by reference in their entirety.

### FIELD OF THE INVENTION

The invention relates to an intervertebral fusion implant for the fusion of two adjacent vertebrae, comprising a main body having first and second legs, adjustable between a flat and an oblique terminal position, and a pivoting blade attached to a free end of the first leg.

### BACKGROUND OF THE INVENTION

The intervertebral disks of the spinal column suffer degeneration as a result of wear or of pathological changes. If conservative treatment by medication and/or physiotherapy is ineffective, surgical treatment is sometimes indicated. In this connection, it is known for a movable or immovable implant to be inserted into the intervertebral space containing the degenerated inter-vertebral disk. This implant takes over the support function of the degenerated intervertebral disk and to this extent restores a stable bearing between the adjacent vertebrae. Immovable implants are also referred to as “fusion implants”.

Various surgical techniques are known for implanting the fusion implants. A traditional surgical technique involves a ventral access route, in order thereby to avoid the danger of damaging the spinal cord in the vertebral column. A ventral access route and avoidance of the spinal column also affords the opportunity to introduce a fusion implant with large cross-sectional dimensions, providing significant support for weight-bearing vertebral bodies. However, these advantages are obtained at the price of a very long access route through the abdominal cavity or thoracic cavity of the patient, and the need to pass closely by the major blood vessels. Since complications can result, an alternative access route has become established, namely from the dorsal direction. Although the latter affords the advantage of a short route, there is a danger of collision with or damage to the spinal cord. To minimize this danger, the operation is usually performed by minimally invasive surgery. Approaches of this kind directly from the dorsal direction or from a more lateral direction are known as PLIF (posterior lumbar intervertebral fusion) or TLIF (transforaminal lumbar interbody fusion), in which the intervertebral disk is exposed from the posterior or lateral direction, respectively. Because of the small transverse incisions used in such an approach by minimally invasive surgery, size of the fusion implants is greatly restricted.

For treatment using the PLIF or TLIF technique, very small fusion implants are known. They afford the advantage of being able to be implanted by minimally invasive surgery thanks to their small size. However, an inherent disadvantage of their small size is that their support function is limited as a result of said small dimensions and is sometimes inadequate. Although a larger size of the fusion implants would improve the support function, this is impractical because of the limits of minimally invasive surgery.

In US 2014/0188224 A1 it has been proposed to design an intervertebral fusion implant for the minimally invasive surgery in such a way that a laterally pivotable side bracket is arranged on a small, for example box-shaped support body. In a position of assembly, the side bracket is retracted into the support body. In this way, the cross section required for the implantation remains limited to that of the support body itself. At the intended site of implantation, the side bracket, which is designed as a kind of toggle expander, is pivoted out to its working position by an actuating instrument. The side bracket is thus spread open laterally. It functions with its bottom and with its top as additional support. Overall, the implant thus provides a support surface approximately in the shape of an isosceles triangle. The support surface is thus considerably increased compared to that of the support body. Against this advantage of the quite large support surface, there is a disadvantage in that adaptation to the anatomical circumstances in the intervertebral space is possible only to a limited extent.

### SUMMARY OF THE INVENTION

An object of the invention is to provide an improved intervertebral fusion implant which, while still having small dimensions, can be better adapted to different anatomical conditions.

Solutions according to aspects of the invention resides in the features of the independent claims. Advantageous developments are the subject matter of the dependent claims.

According to one embodiment, an intervertebral fusion implant for fusion of two adjacent vertebrae, comprises a main body having a first leg, a second leg and an intermediate articulated joint, configured as an V-shape adjustable between a flat V-position and an oblique terminal V-position, a pivoting blade having two ends, one being rotatably attached to an end region of the first leg far from the articulated joint, a guiding trough formed at the second leg, which comprises two lateral side walls and two end-faces, one of the two end-faces being stationary and one being movable with respect to the trough, wherein the moving end-face is formed by a traveller movable along the trough such as to vary its distance to the stationary end-face, and the traveller is rotatably attached to the other end of the pivoting blade, wherein an attachment device for engaging a holding instrument is provided at the stationary end-face, and a second attachment device for engaging a pulling organ is provided at the traveller, the traveller providing an aperture for passage of the holding instrument, and further a lifting mechanism is provided, the lifting mechanism comprising a lifting plate adjustable in height between a retracted state and a raised state in which the lifting plate is spaced apart from a top surface of the legs and forms a bearing for one of the end-plates of the adjacent vertebrae.

The term “flat” V-position shall be understood such as to mean an essentially flat configuration of the two legs forming the adjustable V, particularly having an opening angle between the legs of 170° or more, including 170° to 190°, preferably 175° or more, including 175° to 180°, further preferably substantially 180°.

The term “oblique” V-position shall be understood as that position in which the legs form a proper “V”, in particular having an opening angle of 45° to 150° between the legs.

The term “terminal” V-position shall mean that position in which the implant is laterally expanded such as to function as an intervertebral cage.

Aspects of the invention are based on the concept of providing an improved lateral expansion function which combines advantages of compactness for eased moving of the implant into the intervertebral space with advantages of providing a height adjustable lifting plate for improved adaption to the anatomical requirements in the vertical direction. Owing to the traveller rotatably attached to the other end of the pivoting blade a design which is both, robust and easy to handle, is achieved. Further, it allows optimal fitment to the individual height of the intervertebral space and possibly also individual lordosis angle. In this way, an optimal adjustability and therefore adaptability of the intervertebral fusion implant to the particular anatomical circumstances is achieved. A considerable number of different, conventional intervertebral fusion implants of different widths and in particular of different heights are thus replaced.

As mentioned above with regard to US 2014/0188224 A1, the prior intervertebral implants suffer from the disadvantage that adaptation to the anatomical circumstances in the intervertebral space is possible only to a limited extent. It also has to be noted that while the upper and lower cover surfaces of prior art implant support bodies are designed to bear on the endplates of adjacent vertebrae, said surfaces usually have a planar surface design which cannot be tailored to the corresponding vertebra endplate not having such a planar surface but a concave surface facing the planar cover surfaces of the implant body. This disadvantage also applies to further prior art intervertebral implants, e.g. those disclosed in WO 2011/011609 A2 or EP 2 2 777 633 having an expandable mechanism between both upper and lower surfaces allowing the adjustment of the implant between the end-plates of adjacent vertebrae in height only.

An aspect of the implant, according to some embodiments, is that it can be expanded in both directions-the lateral direction and the vertical direction. While the lateral expansion is provided by the laterally pivotable side bracket arranged on a support body which can be pivoted out to its working position by an actuating instrument, the vertical expansion is provided by the lifting plate. None of said above cited prior art documents provide said both functionalities in conceit, i.e. the option of expanding the implant in the lateral and vertical direction. Having said both functionalities in concert as per the implant as defined in the claims allows for a tailored positing of the implant within the intervertebral space in order to provide the implant's support function at the best.

Furthermore, the applicant has surprisingly noted that the implant comprising an additional lifting plate allows the implant's ingrowth into the intervertebral space much faster compared to implants not having said lifting plate and provides the following explanation thereof:

Each medical implant constitutes a foreign body for the implantee and therefore brings about a complex biological interaction on a very wide variety of different levels. One of the most important reactions of the body is recruitment of osteogenic stem cells to the implant surface, known as osteoconduction. In this process, in a first step, the implant surface absorbs fibrinogen, to which there is attachment of platelets, which on their part release osteogenic growth factors when activated and induce migration of osteogenic stem cells to the implant, specifically the implant surface. The osteogenic stem cells secrete an organic bone matrix, which is mineralized by calcium phosphate deposition. In the ideal case, the implant is tightly joined to the bone following completed osteoconduction, which imparts primary stability, and osteointegration, which imparts secondary stability.

The applicant therefore assumes that implant's faster ingrowth into the intervertebral space compared to implants not having said lifting plate is triggered by the additional surface presented to the osteogenic stem cells once the implant has been implanted within the intervertebral space. It is known that the more implant surface is presented to the osteogenic stem cells the faster the implant gets fused to and integrates within the bone material of adjacent vertebrae. The fusion implant as per the present invention obviously provides via its lifting plate an additional surface said osteogenic stem cells can be adhered to, i.e. the osteoinduction and osteointegration process finds additional support by said lifting plate independent from and in addition to said process on the upper and lower support surfaces of the implant.

Furthermore, it has been surprisingly noted that the lifting plate on the cover surface of the support body reinforces and braces the implant as a whole, in particular once the lifting plate has been raised and is spaced apart from the cover surface of the support body. Due to said stiffening effect by said lifting plate more lateral forces can be compensated by the implant. Furthermore, said additional stiffening by the lifting plate opens the door for a smaller implant size, which is a further advantage, in particular if a less as possible invasive surgery becomes an issue.

In addition, said lifting plate above the cover surface of the support body additionally protects the inside of the support body from external influences, wherein said inside may house for example the lifting mechanism. Said lifting mechanism inside the support body thereby becomes less exposed to external forces or ingrowth of bone tissue. The later might become a major disadvantage, at least for the following two reasons: First, the inner compartment of the support body as well as the elements therein (e.g. a lifting mechanism) may not comprise a biocompatible surface which may trigger irritations and inflammation. Second, if the implant must be removed from the patient's body or replaced by another implant which goes in hand with adjusting the lifting plate in its retracted state prior to its removal from the intervertebral space, then this might become impossible due to damages or blocking of the mechanism. In one embodiment of the present invention the cover surface of the implant body is substantially a closed surface. Said substantially closed surface may still comprise an opening or openings for e.g. the holder(s) of the lifting plate extending through the cover surface into the implant body for connection with the lifting mechanism which might be housed in the interior space of said body.

Furthermore, if the lifting plate has a reduced dimension compared to the implant body's cover surface it is spaced apart in the raised state, then this allows a more tailored installation of the implant's surfaces to the vertebra's end plate and ideally closely follows the vertebra's surface in the attachment region. In other words, the grading from the lifting plate to the support body's surface allows much better to follow the vertebra's non-planar end-plate surface.

In addition, the lifting plate can be angled to the support body, preferably with respect to the lower cover surface of the support body. One major advantage in this respect is that the angled configuration of the lifting plate aids in restoring proper lordosis. In other words, the normal inward lordotic curvature of the lumbar and cervical regions of the spine can be restored by said angled lifting plate thereby providing the patient with a stronger back and curved structure as in healthy persons. The whole lifting mechanism may be angled and/or the lifting plate with its upper surface. In a preferred embodiment of the invention the angle between the lifting plate's surface and the support body, and in particular between the lifting plate's surface and the lower of the lifting plates cover surface is about 2° to about 15°, preferably about 3° to about 11°, further preferably about 4° to about 8°. In a preferred embodiment of the invention said angle corresponds to the normal angle between adjacent vertebrae as it can be found in healthy persons.

However, the invention not only permits a considerable reduction in the number of different sizes and variants of intervertebral fusion implants that have to be kept in stock; in addition, the intervertebral fusion implant according to the invention is also easier to implant. Its width is minimized by virtue of the V-configuration, and its height is minimized by virtue of the adjustable lifting plate, and therefore, by virtue of its small overall size, it can also be easily implanted by minimally invasive approaches with a particularly small transverse incision, yet can be expanded after implantation to provide significant cross-sectional support to the adjacent vertebral bodies. The invention thus combines easy implantation with versatility and the possibility of treating different anatomical configurations

Whereas many different parts are conventionally required for this purpose, an adjustable intervertebral fusion implant according to the invention will in future be sufficient. In addition, by virtue of the adjustable V-configuration and the height-adjustable lifting plate, the invention permits easy implantation owing to small dimensions of the support body in its position of assembly. The expansion has the effect that the main body is transferred from a purely longitudinal configuration into a broad triangle shaped configuration while in the intervertebral space, such that it lies transversely on the anterior aspect of the vertebral apophysis, which is particularly well suited for the transfer of loads. Therefore, quite complex and extensive defects in the area of the intervertebral disk can also be treated by minimally invasive surgery in a manner that is both, safe and easy to accomplish by the operating surgeon.

It is preferred that the dimensions of the first and second legs are selected such that they form an isosceles triangle with the pivoting blade, preferably a right-angled triangle in the terminal V-position.

In order to provide not just an improved implantation but also to allow an improved initial healing-in of the implant, the pivoting blade preferably is made solid. This allows for an improved load bearing capability and further enables the implant to perform as a vessel that is closed all-around. Due to this “closed all-around” feature any bone graft or other ingrowth promoting material placed inside the implant (the space defined by the two legs and the pivoting blade) stays inside that space and can perform its desired function there. There is no risk of unwanted loss due to migration as it was experienced beforehand. In order to further enhance the “closed all-around” feature, preferably a plug is provided for the aperture in order to seal the traveller.

For securing the implant in its terminal V-position which forms said triangle, a locking device is provided. Thereby any unwarranted moving back into a flatter V-position is blocked. Preferably, the locking device comprises a detent, a friction-fit configuration and/or an over-the-center mechanism for the pivoting blade and the traveller.

The lifting plate is preferably configured such that in its retracted state it is flush with a top cover of the first leg. With such a flush or preferably even recessed configuration of the lifting plate on the first leg, the lifting plate merges seamlessly into the outer contour of the first leg. In this way, the lifting plate does not contribute to increasing the size of the outer contour, since it does not protrude. The non-protrusion of the lifting plate also affords the in practice very significant advantage that no additional edges pointing in the direction of implantation are formed, such that the insertion of the implant to its intended site of implantation is, according to the invention, just as easy as with an implant that does not have a lifting plate.

In a preferred embodiment the lifting mechanism comprises a worm gear with worm and worm wheel, and the worm wheel is preferably directly attached to a lifting element of the lifting plate. By such a worm driven mechanism a powerful and yet compact drive for the lifting plate can be realized. Moreover, the worm drive is self-locking against unwanted creeping back of the lifting plate under load. It is not essential for the worm to be designed to remain permanently on the support body. In fact, it is preferably for the worm to be insertable and removable. Provision can also be made that the worm is arranged on an actuating instrument which is delivered from outside (i.e. from outside the body) through an access tube. It then suffices for the worm, with an instrument, to be inserted into the support body in order to actuate the lifting mechanism only when adjustment of the lifting plate is in fact intended. After the lifting mechanism has been actuated, the instrument with the worm can be removed.

Preferably an access hole for driving the lifting mechanism is provided in a wall of the first leg, wherein the access hole is aligned to be in-line with the aperture of the traveller. Thereby a driving instrument for driving of the lifting mechanism can be routed through the same access path as it was used for actuating the traveller in order to transfer the legs of the implant into their terminal V-position. Thereby no additional access path for the lifting mechanism is required, thereby easing surgery action considerably.

The lifting mechanism for the lifting plate can be designed in various ways. In a preferred embodiment, two or more lifting elements are provided for the lifting plate. This affords the advantage that the force needed for the expansion is distributed over different points. Preferably the lifting elements are coupled by a wheel drive in order to ensure synchronous movement. A high degree of reliability can thus be achieved. Alternatively, however, provision can also be made that a central lifting element is provided for the lifting plate, preferably combined with means for preventing rotation. The central lifting element permits a compact structure and a generally simple adjustment mechanism. By the means for preventing rotation, it is also ensured that the lifting plate safely maintains its intended orientation with respect to the support body even in an elevated state. Rotations of the lifting plate, which could possibly lead to irritation in the surrounding tissue as a consequence of rotating corners or edges, are thus effectively avoided.

The invention further relates to an instrument set for said intervertebral fusion implant, wherein the instrument comprises: a holding instrument with an holder device at its front end, an actuating rod insertable into the guide instrument, the actuating rod having an engagement device at its front end and a driving element, in particular a handle, at its rear end, and an adjustment rod insertable into the guide instrument, the adjustment rod having a coupling for a lifting mechanism of the implant at its front end.

By virtue of this instrument set, just a single access path is required for implanting, expanding the implant laterally by moving the traveller in order to establish the terminal V-position, and adjusting the height of the implant by elevating the lifting plate. Requiring just one access path instead of two or more is much less stressful for the patient and it avoids substantial risks inherent to any multi-path technique, in particular finding a second access path which is unobstructed in the vicinity, and further ensuring that both access paths meet at the desired position. It is thus a big advantage that the invention allows a single path access, in terms of speed and ease of operation, lower stress for the patient as well as lower risk, in particular of irritating or even injuring nerve pathways running along the spinal column.

The single access path is suitable to be performed by minimal invasive surgery. Being rather small, the access path can be chosen straight, which facilitates access to the implant site. In particular it allows usage of robust instruments in order to achieve a long-term stable positioning of the implant. Preferably, a rear end of the holding instrument is configured with a hammerhead for accepting hammer-blows.

For ensuring proper access and for ease of transporting the implant into the intervertebral space, the holding instrument is of a multi-part construction. It comprises a guiding tube as an outer part and a hollow guide rod as an inner, through which all other instruments are to be guided. By this configuration a stationary access path is established, allowing transport of implant into its position as well as change of instruments without any risk of irritating surrounding tissue. Further, by means of the guide rod the implant is firmly attached to the holder, allowing positive control of the implant regarding its movement into and positioning within the intervertebral space. After implantation, the guide rod is released from the implant and removed out of the guiding tube, and eventually the guiding tube will be removed.

Yet further, the invention also relates to an alternative instrument set for handling said intervertebral fusion implant. Like the instrument set described above, the alternative instrument set comprises a holding instrument with an holder device at its front end, an actuating rod insertable into the guide instrument, the actuating having an attachment port at its front end and a driving element, in particular a handle, at its rear end, and an adjustment rod insertable into the guide instrument, the adjustment rod having a coupling for a lifting mechanism of the implant at its front end. However, some of the instruments are configured differently, as will be explained in further detail below.

In the alternative instrument set, the holding instrument is configured as a guiding plier, comprising a guiding tube and an actuating handgrip with at least one movable handle having a gripping portion and an action lever portion, and an engagement device for attaching the implant at a front tip of the guiding tube. Preferably, the movable handle is detachable and/or said action lever portion is provided with a coupling connectable to other instruments. By configuring the holding instrument like a plier improved handling characteristics could be achieved. The handle provides superior positional and directional control of the holding instrument and the implant attached thereto. For attaching of the instrument, a hollow guide rod is provided and mounted inside the guide tube, its front end engaging the implant, preferably by a threaded connection, and pulling the implant against the front end face of the guide tube. The guide rod can be of such length to span the full length of the guide tube, but preferably it is shorter and is configured to abut against a shoulder provided in a mid-section of the guide tube. For tightening, the guide rod is preferably provided with a tool coupling at its rear end, e. g. a receptacle for a hex socket. After tightening the tool may be removed and as a result the implant is firmly attached to the holding instrument.

By virtue of the detachable configuration the movable handle could be removed in order to feed other instruments into the hollow guide tube. Thereafter the movable handle can be reattached, and the coupling engaged with the instrument if desired. For proper engagement with the coupling, the actuating rod and/or adjustment rod are preferably provided with a connecter for attaching to the coupling. Further, the connecter preferably is configured for transmission of axial forces. This is preferably accomplished by configuring the coupling and the connector for a form-fit, preferably by means of a pair of projections in opposing relation and a matching recess. The opposing projections are engageable in the recess, thereby creating a form-fit connection. By virtue of such interaction between the coupling at the handle and the engaged connector at the actuating rod or adjustment rod, axial movement and position of the actuating rod or adjustment rod could be controlled by manipulation of the movable handle. In the case of the actuating rod, actuation of the movable handle pulls the actuating rod backwards and thereby the two legs of the implant are transiting from the initial flat V-position to their terminal oblique V-position. The V-position transit can be accurately manipulated by exerting the movable handle. Correspondingly, in the case of the adjustment rod the handle will be immobilized, preferably by arresting means, in order to axially lock the adjustment rod (however, owing to the design of the coupling/connector arrangement rotational mobility will be retained).

It is particularly preferred that the coupling and connecter are releasable in at least two different positions of the handgrip, a relaxed and a squeezed position. Thereby the coupling could be released also after activation of the movable handle and of the action lever, i. e. after actuation of the actuation rod or the adjustment rod. This allows—e.g. in the case of the actuation rod—a release of the coupling and as a result removal of the actuation rod subsequent to the transition of the implant from the flat V-position to its oblique terminal position effected by activation of the actuating rod by manipulation of the handle. Similarly, once the height of the implant had been adjusted, the coupling could be disengaged thereby allowing removal of the adjustment rod while the implant maintains its elevated state.

It is preferred to provide an arresting device for the handle configured for arresting the handle. Thereby the movable handgrip is immobilized, blocking any unwarranted squeezing of the handle. This is particularly useful for forced manipulation of the holding instrument, i.e. by hammer blows to the holding instrument itself or any other instrument attached to the holding instrument. Further, by virtue of such an arresting device the coupling at the action lever of the movable handgrip maintains its position, thereby providing a stable reference. This is particularly useful for usage of the adjustment rod, thereby allowing positive control of the achieved elevation of the lifting plate of the implant. The arresting device may be configured such as to be auto-engaging once the handle reaches a wide open position.

Preferably, the connector on the adjustment rod is offset forward compared to the connecter on the actuating rod. Since the connecter is engaged by the coupling on the handle, a different position of handle will result dependent on the type of rod inserted. Thereby a handle position can be selected which is most convenient for the relevant task (actuating, adjusting). Further, by selecting the wide open position appropriately it could be achieved that the arresting device will auto-engage if one of the rods, e.g. the adjustment rod is inserted, but is not engaged if the other of the rods is inserted. Thereby operation of the instrument is simplified for the surgeon and less prone to usage errors.

Further, a crank is preferably provided which is configured to be releasable connected to a rear end of the adjustment rod. Rotating the adjustment rod requires a lot of torque due to counter forces exerted by raising of the lifting plate against the ligaments of the spinal columns. The required torque may be too high for a reliable actuation by hand of the surgeon. In order to provide such a reliable actuation even under difficult circumstances, the crank is provided which provides—referenced to the same actuation force provided by the surgeon—a much higher torque. The crank thus acts as an easy to use torque amplifier. The crank is preferably configured for manual operation. Thereby the surgeon still retains a positive tactile feedback of the raising action exerted by actuation of the adjustment rod.

Preferably, indicator devices are provided for the actuation and/or adjustment rod, the indicator devices comprising a scale and an marker, one of which being stationary with respect to the guiding tube and the other being stationary with respect to the actuation/adjustment rod. It is further preferred that an observation window is provided on the guiding tube, which provides visual access to the actuation/adjustment rod positioned in the guiding tube.

Further preferably, the engagement device is a thread provided at a tip of the guide tube. Thereby a direct and robust engagement of the implant to the holding instrument can be achieved. Further, at a front face of the guiding tube at least one fixation projections are provided which are configured to interact in a form-fit manner with the implant. Thereby any unwarranted rotation of the implant relative to the guide tube is avoided.

Preferably, a worm drive is provided at a tip of the adjustment tool, and further preferably a center bearing pin is provided at the tip. By virtue of the worm drive a direct, reliable and yet compact actuation of the height adjustment mechanism of the implant. The center bearing pin keeps the worm drive centered and thus reduces parasitic fraction which otherwise could be induced by misalignment. Further, the center bearing pin concentrates any friction forces to an area close to the center, and owing to the small or nil off-center distances the friction momentum is kept very low.

Further, either instrument set preferably comprises a trial tool. It comprises an elongated main body having a trial implant attached at its front end an actuating handle at its rear end. The trial implant may form an integral part of the trail tool or could be realisably coupled in order to be exchanged. The trial implant is of the same general constitution as the implant described, although it preferably is thinner (lesser height) and further preferably is devoid of a lifting plate and mechanism. However, the trail implant features the V-configuration and—in an idle state of the tool—is in the flat V-position. Thereby it forms a line with the main body of the trial tool, which preferably has the same thickness as the trial implant. The actuating handle is coupled to the trial tool in such a way, preferably by a push rod, as to actuate the trail implant so that it is brought into the terminal oblique V-position. By reverse action of the actuating handle the trail implant returns into its flat V-position, thereby facilitating removal of the trial tool.

The trial tool preferably features at least one of a depth scale and a position indicator scale. The depth scale is preferably attached in a front region, just rearwards of the trial implant. Its scale is configured such as to show depth of insertion of the trial implant; the scale is preferably calibrated in millimetres or fractions of inches. The position indicator scale is operationally coupled to an indicator affixed to the actuating handle and/or the push-rod. By virtue of this the actual V-position of the trial implant as a result of handle actuation will be displayed for visual reference.

The invention further comprises a set or kit of parts comprising the intervertebral fusion implant of the present invention and the instrument set for the intervertebral fusion implant as outlined above.

### BRIEF DESCRIPTION OF THE DRAWINGS

The invention is explained in more detail below with reference to the attached drawing in which advantages illustrated embodiments are depicted:

FIG. 1 shows a schematic view of an intervertebral fusion implant placed into an intervertebral space between adjacent vertebral bodies;

FIG. 2 shows implant views of the implant in the position of assembly, intermediate position, and terminal position;

FIG. 3 shows perspective views of the implant in terminal and locking positions;

FIG. 4 shows an exploded view of the implant;

FIG. 5 shows a view of an instrument set;

FIG. 6 shows a detail of attachment of an insertion instrument to the implant;

FIG. 7 shows a detail of the attachment of a spreading instrument to the implant;

FIG. 8 shows an actuating instrument and a coupling ready for attachment thereto;

FIG. 9 shows a height adjustment together with the coupling and an engaged state;

FIG. 10 shows a variant for the instrument set;

FIG. 11 shows an actuation rod and a holding instrument of the instrument set of FIG. 10 in an initial and actuated state;

FIG. 12 shows an adjustment rod and the holding instrument of the instrument set of FIG. 10 and a side view of a detail;

FIG. 13 shows a side view of an intervertebral space with the implant in its elevated state; and

FIG. 14 shows a trial tool for the instrument set of claim 10.

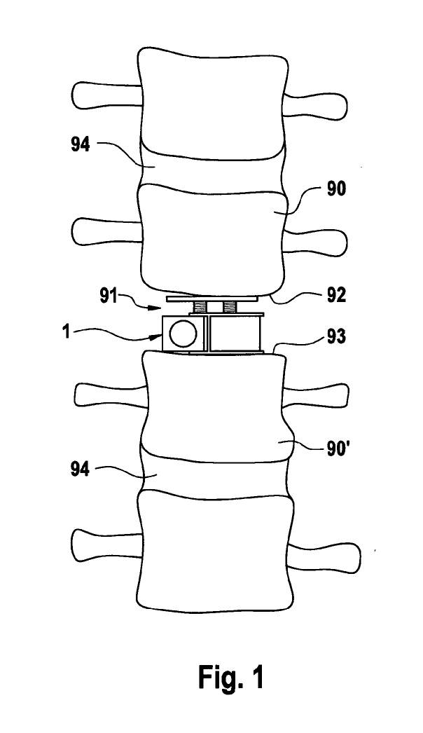

### DETAILED DESCRIPTION OF THE INVENTION

An illustrative embodiment of an intervertebral fusion implant according to the invention is in its entirety designated by reference no. 1. It is provided for implantation in an intervertebral space 91 between two immediately adjacent vertebral bodies 90, 90′.

In the physiologically intact vertebral column, an intervertebral disc 94 is located in the intervertebral space between the vertebrae. This intervertebral disc 94 may undergo degeneration as a result of disease or wear, resulting in a need to at least partially resect the intervertebral disc 94. For providing the sufficient support in the intervertebral space 91 despite loss of intervertebral disc material, and to prevent collapsing of the vertebral column, the intervertebral fusion implant 1 is inserted into the intervertebral space 91. It provides a supporting function in lieu of the removed intervertebral disc and further facilitates fusion of the adjacent vertebrae 90, 90′ in a natural way by bone growth.

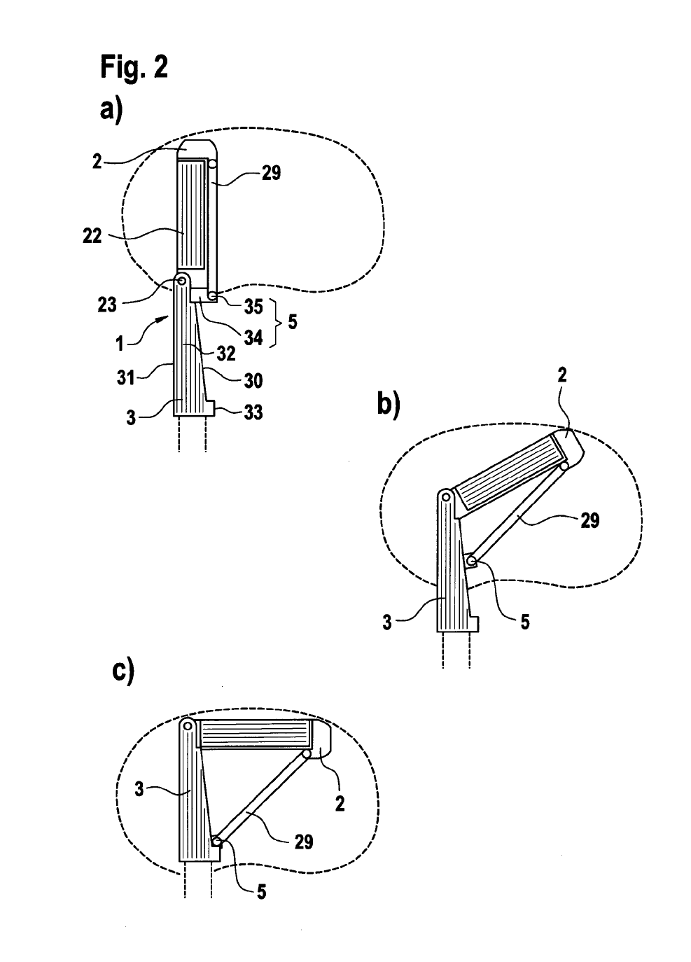

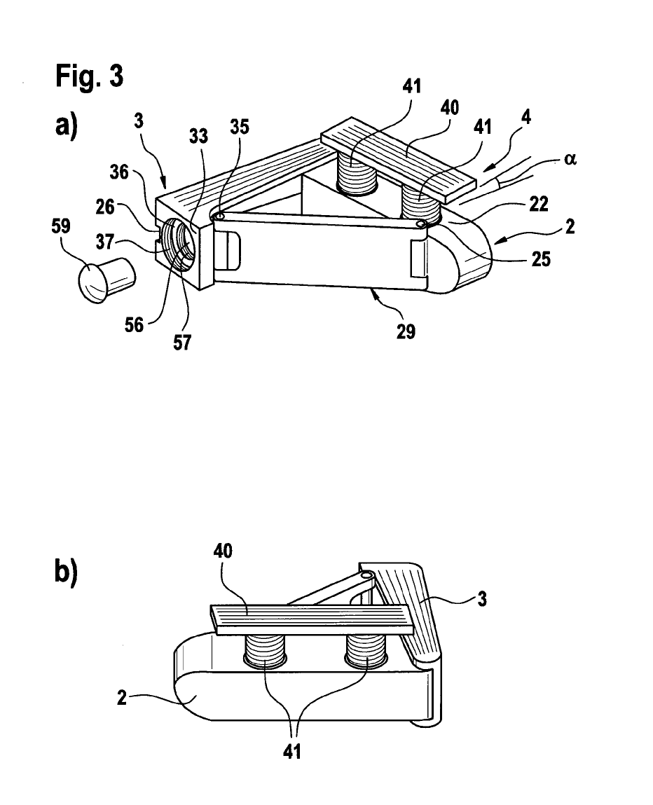

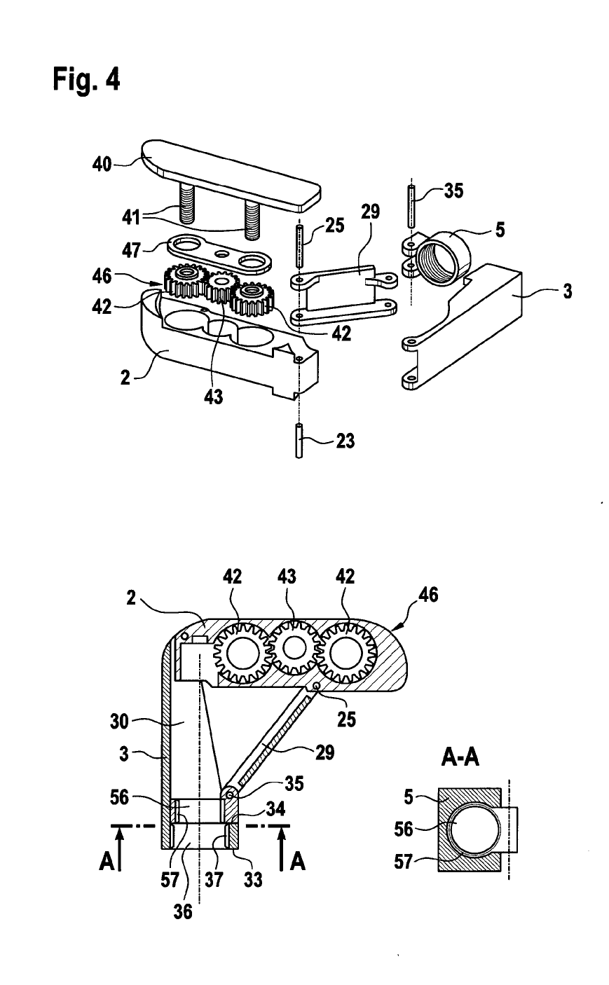

Structure and function of the fusion implant are explained in the following base on the illustrative embodiment, with particular reference to FIGS. 2 to 4. The intervertebral fusion implant comprises a main body having a first leg 2, a second leg 3 connected by an intermediate articulated joint 23. It is configured as being of an adjustable V-shape between the flat V-position and an oblique V-position. Attached to an end region of the first leg 2 far from the articulated joint 23 is a first end of a pivoting blade 29, by means of a first pin joint 25. The pivoting blade 29 is attached with its other end to a traveller 5 by means of a second pin joint 35.

The second leg 3 is configured such as to form a guiding through. It has one open side which is the lateral side of the second leg 3 facing towards the first leg 2 in an oblique V-position (see FIG. 2 C). The guiding through 30 further comprises two-end-faces, first being a stationary end-face 33 positioned at an end-region of the second leg 3 which is far from the articulated joint 23. The other end-face is a movable end face 34 which is enabled of a sliding movement along the guiding through 30 in direction towards the stationary end-face 33, thereby varying the distance between the two end-faces 33, 34. The moving end-face 34 is formed at the traveller 5 to which the second end of the pivoting blade 29 is attached in a rotatable manner by means of the second pin joint 35.

The far end of the second leg 3 is formed by the stationary end-face 33 of the guiding through 30. In that stationary end-face 33, a through-hole 36 is formed having a holding thread 37 formed on its inner surface. It acts as a passageway in order to reach the inner space of the guiding through 30 from the outside, and to reach from the outside to the traveller 5 sliding along the guiding through 30. The traveller 5 is similarly equipped with a second through hole 56 which comprises an actuating thread 57 on its inner surface. Both through-holes 36, 37 are positioned such as to be aligned with each other, in other words they are concentric. The passageway created by the through-holes 36, 37 can be sealed by insertion of a plug 59 from the exterior side of the stationary end-face 33.

At a lateral side of the second leg 3 facing the way from the first leg 2 in an oblique V-position an arresting notch 26 is formed at the edge to the front face 33. The arresting notch 26 is formed such as to accept a tongue 61 of a holding instrument 60 such as to avoid relative rotation between the implant and the holding instrument, as it will be explained in further detail later on.

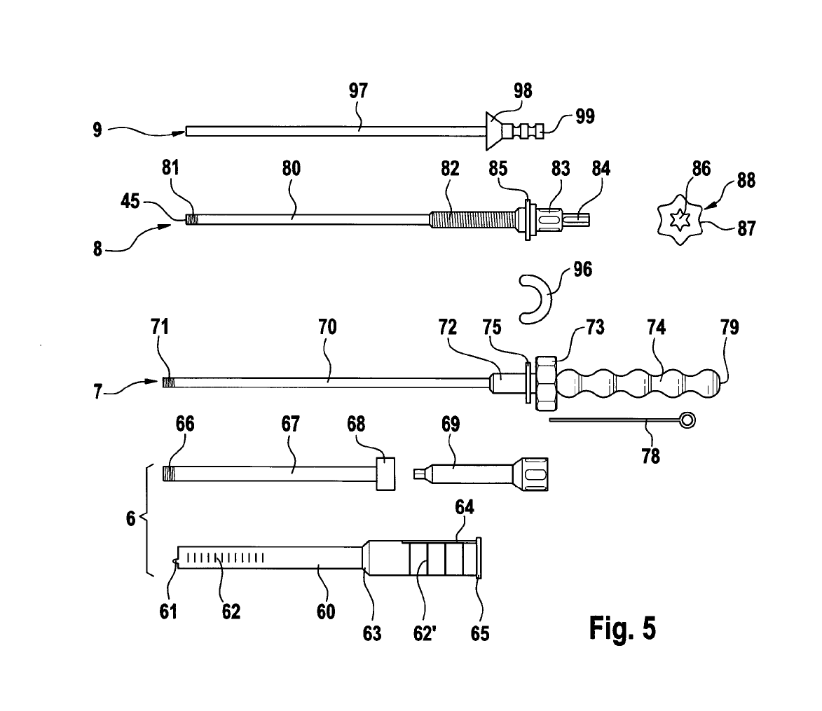

The first leg 2 is further provided with a lifting plate 40 which forms a top cover of the first leg 2. The lifting plate 40 is actuated by a lifting mechanism 4, which comprises two lifting spindles 41 and two driven wheels 42 fitted on the lifting spindles 41. The driven wheels 42 are provided with an inner thread for screwing onto the lifting spindle 41. Both driven wheels 42, each for driving its associated lifting spindle 41, are interconnected by means of an intermediate wheel 43. By virtue of this intermediate wheel 43 it is ensured that both driven wheels 42 are rotated in the same manner and, as a result, the lifting spindles 41 will be elevated by a same height. The driven wheels 42 and a worm spindle 45 (s. FIG. 5) form a worm drive 46. It is accessible from the outside of the first leg 2 via an actuation opening 45 in a lateral side of the main body of the first leg 2. The worm drive 46 is arranged in the interior of the main body of the first leg 2 and is provided with a covering 47. The worm drive 46 functions such that the worm spindle 45 is rotated by means of an actuating instrument which is moved through the through-holes 36, 37 and the guiding through 30 into engagement with one of the driven wheels 42 of the worm drive 46. It is to be noted that the worm spindle 45 may form a separate part or may be integrated into the actuating instrument 8 namely such that the actuating instrument 8 is provided with a worm spindle 45 at its front end. Thereby, by turning the actuating instrument 8 with the worm spindle 45 the driven wheels 42 rotate, whereby one of the driven wheels 42 being arranged closer to the joint 23 is being driven directly and the other driven wheel 42 is being driven indirectly by means of the intermediate wheel 43. Due to rotation of the driven wheels 42 the lifting spindles 41 are moved upward raising the lifting plate 40. The lifting plate 40 is thus elevated, the amount of elevation being defined by the number of revolutions of the actuating instrument 8. By this, a stepless fine adjustment of the height of the lifting plate 40 and therefore of the complete implant 1 can be accomplished. Said lifting plate 40 can be angled to the support body 2 as indicated by the angle “α”. Said angle α can be about 2° to about 15°, preferably about 3° to about 11°, further preferably about 4° to about 8°. Said angle may correspond to the normal angle between adjacent vertebrae as it can be found in healthy persons at the intervertebral fusion site.

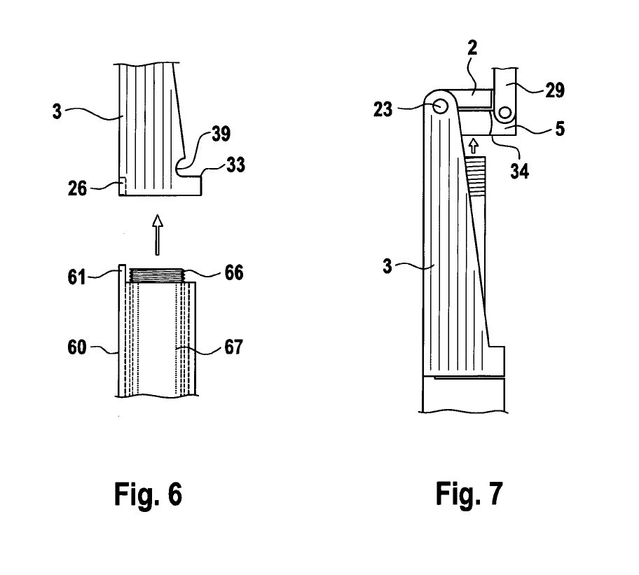

FIG. 5 shows an overview of an instrument set according to an exemplary embodiment of the present invention. It comprises a holding instrument 6, an expansion rod 7, an adjustment rod 8, a coupling 96, a torque amplifier device 88 and a plunger 9. The holding instrument 6 is a multi-part construction and comprises a hollow holding tube 60 as an outer part and a hollow guide rod 67 as in inner part, and an actuating socket 69. The holding tube 60 is of an elongated shape having a tubular front section carrying scale markings 62 on its surface. The scale markings are adapted such as to indicate insertion depth. Further, at its front end a tongue 61 is provided which is projecting outwards and designed to interact with a complementary shaped notch 26 at the second leg 3 of the implant. The holding tube 60 comprises at its rear end region an enlarged diameter with a shoulder 63 connecting its rear end region with the increased diameter with the tubular front section. On the outer surface of the rear end region circumferential markings 62′ are provided for indicating progress of expansion/adjustment, which will be explained later. At its rear end the holding tube 60 is provided with a ring 65 having a further increased diameter. In its interior space the holding tube 60 features a generally funnel shape due to a reduction in diameter from the rear to the front region. The hollow guide rod 67 is dimensioned such as to fit into the interior space of the holding tube 60 and its other dimension is matched to the interior dimension of the tubular front region such as to be rotatably received. The guide rod 67 is provided with a first thread 66 at its front tip, configured for engaging the thread 37 at the end-face 33 of the second leg 3 of the implant 1. At its rear end the guide rod 67 is provided with a collar 68 being configured for accepting a hex-type driving socket 69. It can be releasedly engaged to the collar 68 of the guide rod 67, thereby allowing a rotation of the guide rod by driving the socket 69 with a rotational movement. The distance between the thread 66 at the front end and the collar 68 at the rear end of the guide rod 67 is dimensioned such that upon insertion of the guide rod 67 into the holding tube 60 the collar 68 of the guide rod 67 abuts against the funnel section 63 when the thread 66 at its front end emerges at the tip of the holding tube 60 (see FIG. 6).

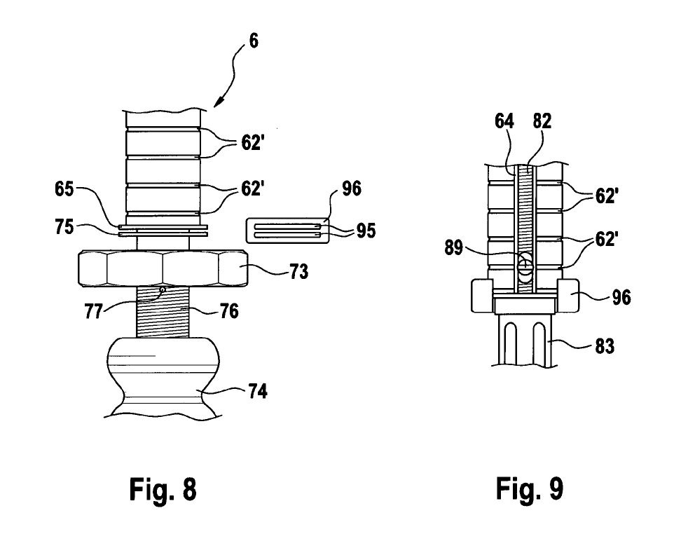

The expansion rod 7 comprises an elongated shank 70 having a second thread 71 at its front end and having a thickened sleeve portion 72 in its rear region being fitted with an annular ring 75 dimensioned such as to having the same outer diameter as the ring 65 of the holding instrument 6. The diameter of the shank 70 is dimensioned such that it is insertable into the hollow guide rod 67 of the holding instrument 6. The sleeve 72 carrying the ring 75 is equipped with a rotary handle 73 at its rear end. The sleeve 72 is rotatably mounted on a third thread 76 at the rear portion of the shank 70 (see FIG. 8). A handle 74 is attached to the rear end of the threaded portion 76. Further, a check hole 77 is provided in the third thread 67, and a check pin 78 is provided that could be inserted into the check hole 77.

The thread 71 at the front end of the shank 70 is configured for engaging the thread 57 provided at the inner surface of the second through hole 56 positioned in the traveller. By engaging the threads 71, 57 a pulling action can be exerted by the expansion rod 7, thereby moving the traveller 5 in a sliding motion from its initial position adjacent to the first leg 2 along the through 30 towards the stationary end-face 33, thereby putting the first leg 2 in an oblique angular relation with the second leg 3 such as to receive its terminal V-position (see FIG. 2a, b and c). The degree of expansion is controlled by turning the rotary handle 73 in a clockwise direction. In order to keep a tight relationship between said expansion rod 7 and the holding instrument 6 into which it is inserted, a coupling 96 is provided which is generally U-shaped and comprises two parallel inner grooves 95. Their width is selected such that they fit snugly over the rings 65 and 75 located at the rear end of the holding instrument 6 and at the expansion rod 7, respectively. The coupling 96 is moved over these rings 65, 75 such that the rings are seated within the grooves 95. Thereby, the axial position of the expansion rod 7 in relation to the holding instrument 6 is affixed. For increasing the expansion state of the implant, the rotary handle 73 is turned in a clockwise direction. For decreasing the expanding state, the rotary handle 73 is to be turned in counter-clockwise direction. By virtue of the coupling 96, it is ensured that the expansion rod 7 retains its axial position and does not wander off in a rearward direction in an unwarranted manner during turning of the rotary handle 73, in particular in an anti-clockwise direction. As a result, positional accuracy is much increased.

For checking the expansion state of the implant, a check hole 77 is provided. Once the check hole 77 becomes visible on the thread 76 behind the rotary handle 73 it is assured that the traveller 5 has been moved far enough towards the stationary end-face 33 such that the first and second legs 2, 3 have reached the terminal V-position (see FIG. 2c). The presence of the check hole 77 behind the rotary handle 73 can be verified visually or by employing of the checking pin 78. When it can be pushed into the checking hole 77 the implant is expanded sufficiently.

Once the terminal V-position has been reached, the expansion rod 7 can be disengaged by turning the handle 74 in an anti-clockwise direction, thereby removing the second thread 71 from its engaged position within the thread 57 in the second through hole 56. Thereafter, the expansion rod 7 can be pulled out of the holding instrument 6.

The terminal V-position is maintained by a locking device which is configured as a cutout 39 at the terminal position of the traveller 5 (s. FIG. 6). The cut-out 39 is dimensioned such as to provide a form-fit for the pin of the pin joint 35. Thereby, the traveller 5 is arrested in its position and any unwarranted backward movement of the traveller 5 is blocked.

The rear end of the handle 74 may be equipped with a hammerhead 79. Thereby, insertion of the implant could be facilitated by applying hammer blows on the hammerhead 79 of the handle, thereby pushing the implant 1 forward.

Further, for height adjustment an adjustment rod 8 is provided. It comprises an elongated shank 80 having a worm spindle 45 for the worm drive 46 attached to its front tip. At a rear portion of the shaft 80, a fine pitch thread 82 is provided. At its rear end, further a collar with a ring 85 shaped like the ring 75 of the expansion rod 7 is attached and further rearward a second rotary handle 83 and a machine coupling 84 are provided. After removing the expansion rod 7, the adjustment rod 8 is inserted into the holding instrument 6 and moved forward so far until the worm spindle 45 at the front tip of the shank 80 engages with one of the worm wheels 42. Thereby, the worm drive 46 is completed. By turning the adjustment rod 8 the worm drive 46 is actuated, leading to a rotation of both driven wheels 42 and its intermediate wheel 43, thereby effecting a lifting action of the spindles 41.

As a result, the lifting plate 40 is raised from the top of the first leg 2. In order to ensure the adjustment rod 8 does not move out of its engagement position with the driven wheels 42, the coupling 96 is attached onto the rings 65 of the holding instrument 6 and 85 of the adjustment tool, thereby locking its axial position.

In order to determine the elevation position of the lifting plate 40, an indicator 89 is provided. The indicator 89 is configured as a sliding block having a check mark on its top surface which is placed on the thread 82 and is guided along an axial slit 64 formed at the enlarged diameter portion of the holding tube 60. The check mark on the slider 89 provides an indication for the height adjustment achieved compared to the scale provided by marker rings 62′ on the holding tube. Thereby, it can be positively determined whether the desired height adjustment had been achieved.

For turning the height adjustment rod 8, a rotary handle 83 is provided. In order to achieve more torque a torque amplifying device 88 is provided. It is generally in a star-shaped form and has an aperture 86 at its centre which is shaped in a complementary manner to the rotary handle 83 or the coupling 84. By attaching this device a better grip for the surgeon can be achieved, thereby increasing the torque the surgeon can apply by acting on the device 88 and the adjustment rod 8.

After achieving the desired height, the torque amplifying device 88 as well as the coupling 96 are removed and the adjusting rod 8 will be taken out of the holding instrument 6. The implant is now fully configured.

In order to promote bone ingrowth, bone graft material may be supplied to the implant. To this end, a certain amount of bone graft material is placed into the interior space of the holding tube 60. Its funnel shape facilitates placing of the bone graft material and moving it through the hollow tube 60 and through the through hole 36 at the end-face 33 and the second through hole 56 of the traveller 5 into the inner area of the implant, delimited by a triangle formed by the first leg 2, the second leg 3 and the pivoting blade 29. In order to move the bone material into this inner space of the implant, a plunger 9 is provided. It features an elongated rod 97 shaped such as to pass through the inner space of the hollow tube 60 and thereby to push the bone graft material through the tube into the inner space of the implant. The depth of the plunger is limited by a stopping collar 98 arranged at a rear end and in vicinity of a handle 99 of the plunger.

The pivoting plate 29 is made of a massive construction having a closed surface. Thereby, the bone graft material transferred into the inner space of the implant is kept there and does not wander out into the surrounding tissue.

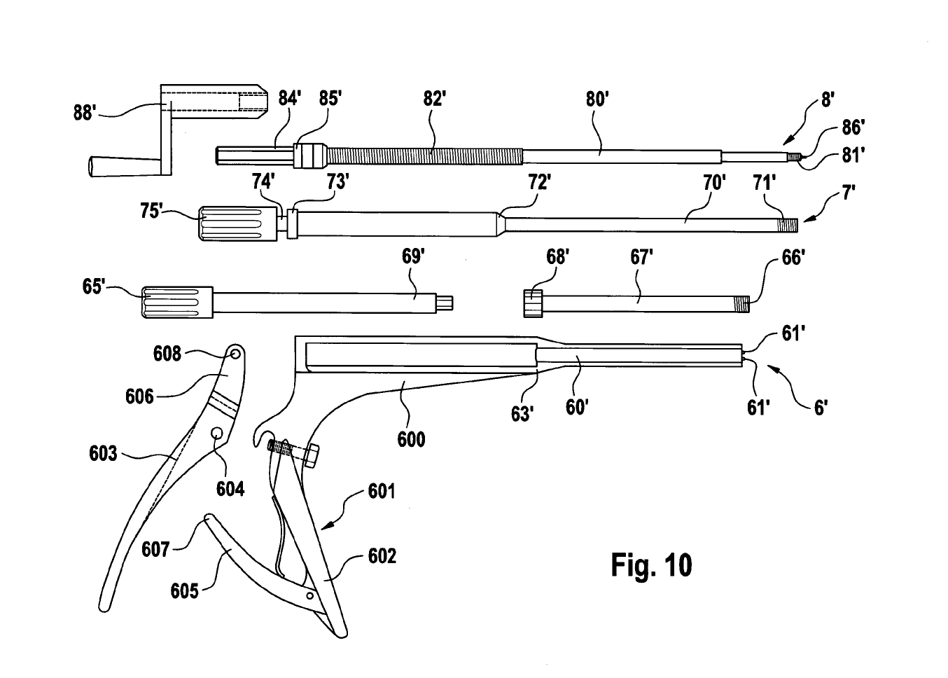

An overview of a variant of the instrument set is shown in FIG. 10. In its general functions it is similar to the instrument set as shown in FIG. 5. Elements having same or similar functions are bearing the same reference numeral, and for further details the explanations given above can be consulted. The variant of the instrument set comprises a holding instrument 6′, an expansion rod 7′, an adjustment rod 8′ and a torque amplifier device 88′.

The holding instrument 6′ is configured as a plier, comprising a handgrip 601 attached to a rear portion of a main casing 600 and a guiding tube 60′ formed at a front portion of the main casing 600. The handgrip 601 comprises a stationary handle 602 which is formed unitary with the main casing 600 and a movable handle 603 being pivotally mounted to the stationary handle 602 by means of a pivot joint 604 located at its middle portion. The pivot joint 604 is releasable such as to allow disengagement of the movable handle 603 (see FIG. 10). The movable handle 603 comprises an action lever 606 at its upper portion with a pair of projections 608 as a coupling.

Between the stationary handle 602 and the movable handle 603 a spring loaded arresting arm 605 is formed. Due its spring loading it exerts a spreading force on the two handles 602, 603. The arresting arm 605 is configured with a hybrid tip 607 which comprises a rounded portion and an angled portion. In the normal open and squeezed state of the handgrip 601 the rounded portion of the hybrid tip 607 slides along an interior facing surface of the movable handle 603, thereby exerting an opening force to the handgrip 601 due to the spring loading. The handgrip thus wants to re-open which is a convenient counter-force to squeezing.

However, in an overextended position of the handgrip 601 the angled portion of the hybrid tip 607 interacts in a wedge like manner with the interior facing surface of the movable handle 603, thereby immobilizing the movable handle 603 blocking any squeezing action. As a result of the blocked movable handle 603 the projections 608 located at the action lever 606 are secured in their position, and therefore provide a stable reference for any instrument placed into the guiding tube 60′ and being in contact with said projections 608. The blocking of the movable handgrip 603 may be released by manually moving the arresting arm 605 out of contact with the movable handgrip 603, thereby allowing the handgrip 601 to return from its overextended position by gentle squeezing.

On a front section of the guiding tube 60′ scale markings 62 are provided. They are configured such as to indicate insertion depth of the implant into the intervertebral space. Further, at a front tip of the guiding tube 60′ a set of four tongues 61′ are provided, each of which projecting outward in a forward direction. The tongues 61′ are configured such as to interact with complementary recesses 26 at the second leg 26 of the implant.

On a rear end section of the guiding tube 60′ a shoulder 63′ is formed which is a front end of an enlarged width section in the main casing 600 forming an extension of the guiding tube 60′ towards the rear. The shoulder 63′ provides a reference position for instruments placed into the main casing 600. Further, an observation window 64′ is provided along a lateral side of the main casing 600, said window providing visual access to the interior of the main casing 600 with said extension. In vicinity of a rim of the observation window 65′ a scale 62′ provided. It is configured for interaction with a marking 89′ on instruments placed into the main casing 600, thereby indicating how far forward the instrument is advanced in a direct and easy to read manner.

A hollow guide rod 67′ is provided and is dimensioned such as to fit into the guide tube 60′. The guide rod 67′ is provided with a first thread 66′ at its front tip, configured for engaging the thread 37 at the end-face 33 of the second leg 3 of the implant. At its rear end the guide rod 67′ is provided with a collar 68′ configured for receiving a hex-type driving socket of a driving tool 69′. The diameter of the collar 68′ is larger than the interior diameter of the guide tube 60′, and thus abuts against the shoulder 63′ in its most forward position, thereby providing a well-defined datum for its position and of the position of the implant attached to the thread 66′ at its front tip. The lengthwise dimension of the guide rod 67′ is selected equivalent to that of the guide rod 67 of the first instrument set described above.

By turning of the guide rod 67′ by means of the driving tool 69′ the implant will be tightened against the front surface of the guide tube 60′, thereby affixing it. In this tightened position, the set of tongues 61′ engages the complementary recesses 26, thereby preventing any rotation of the implant relative to the holding instrument 6. For effecting an easy tightening, the drive tool 69′ is equipped with a handle at its rear end.

The lateral sides of the front end portion of the guide tube 60′ recessed, so that the width of the holding instrument 6′ at its front tip is reduced. Owing to the reduced width an increased space is created for accommodating a protruding rim of any of the adjacent vertebrae between which the implant is to be placed (s. FIG. 13). Further, the reduced width increases visibility of the implant in X-ray imaging made during surgery. In order to further increase visibility of the implant in X-ray images, the tongues 61′ are placed in upper left and right as well as lower left and right corners, in order to provide a clear visual (and X-ray) path in vertical and horizontal direction.

The expansion rod 7′ comprises an elongated shank 70′ having a second thread 71′ at its frond end. Further it features a thickened portion in its rear portion with a tapering section 72′. At its rear end the expansion rod 7′ is equipped with an annular ring 73′. It is configured to bear against a rear end face of the main casing 600 of the holding instrument 6′. Rearward of the annular ring 73′ an actuation handle 74′ is provided, and between these elements an annular ring 73′ is formed. The annular ring 73′ is configured such as to be engageable by the projections 608 of the action lever. Thereby, by squeezing of the handle 601 the expansion rod 7′ can be moved back and forth. For usage, the expansion rod 7′ is fed through the hollow guide rod 67′ until its second thread 71′ emerges at the tip of the guide rod 67′, and then by turning of the actuation handle the second thread 71′ is affixed to the second thread 57 located at the traveller of the implant. For actuation of the implant, the expansion rod 7′ is pulled rearward in order to move the legs from its original flat V-position (used for moving the implant into its implantation site) to the oblique terminal V-position. This pulling action is achieved by squeezing of the handle 601 of the holding instrument. Due to the squeezing, the movable handgrip 603 is moved and its action lever 608 with the projections 609 thereon will be moved backwards, thereby pulling the expansion rod 7′ rearwards by virtue of the projections being engaged in the annular recess 74′. The handgrip 601 allows the surgeon a smooth and well controlled expansion action.

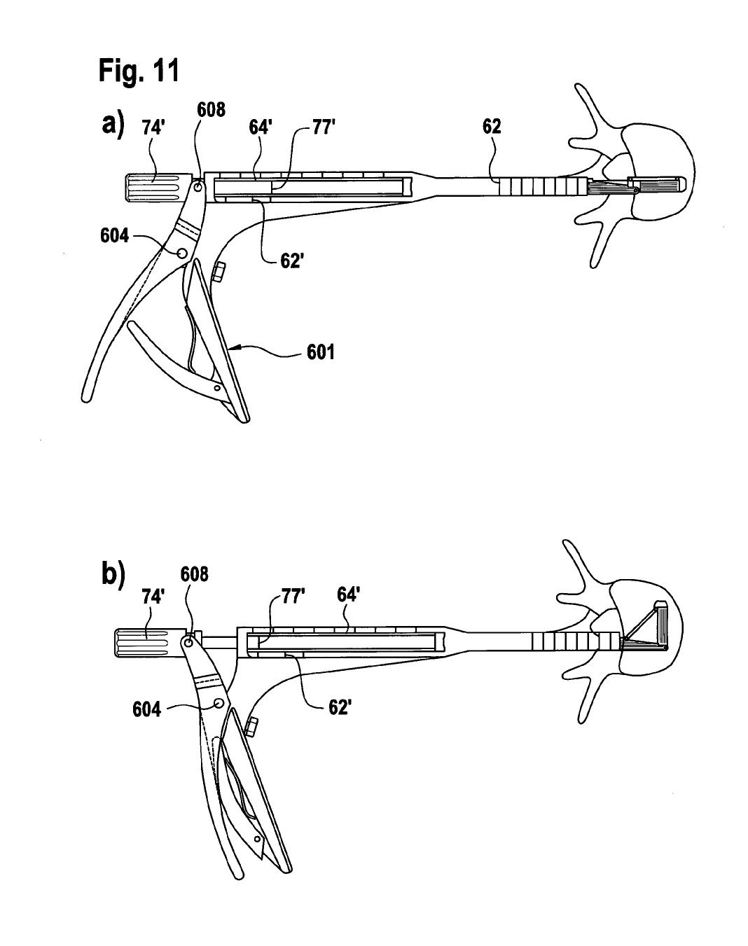

In order to gauge the expansion state reached by the implant, a marking 77′ is formed at the expansion rod 7′, the marking 77′ being positioned such as to be at a zero mark of the scale 62′ in a non-squeezed position of the handle 601, i.e. when the implant is still in its flat V-position (s. FIG. 11a). The marking 77′ will reach a 100% marking (“1/1”) when the expansion rod 7′ is pulled so far backwards such that the traveller has reached a position consistent with the terminal oblique V-position of the implant (s. FIG. 11b). After the desired V-position of the implant has been reached the expansion rod 7′ is removed from the holding instrument 6′.

It is to be noted that the general action of the holding instrument is to expand the implant by squeezing of the handgrip 601. Closure rate will be controlled by braking action exerted by the arresting arm 605, and the friction created by sliding of the hybrid tip 607 on the interior surface of the movable handle 603.—However, in case any need to reverse expansion should arise, this can be easily accomplished by moving the movable handle 603 in the reverse direction. In this case, the stationary handle 602 provides a convenient counter force required for this action. Owing to the scale 64′ even in such a scenario the actual V-position of the implant could be accurately determined.

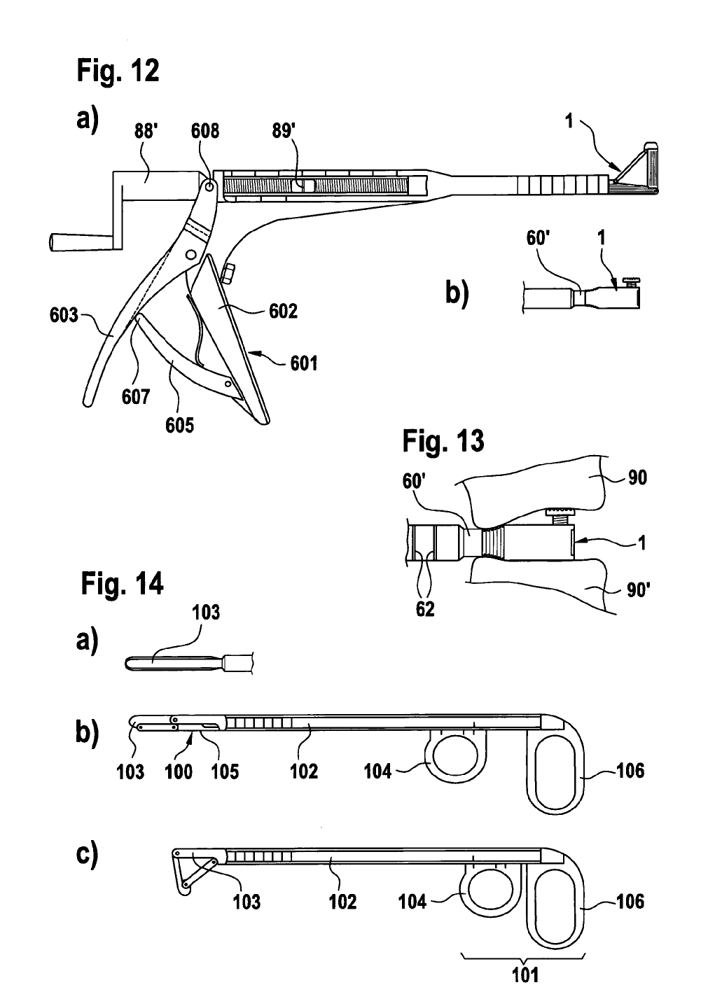

For adjusting height of the implant by raising the lifting plate 40 of the implant the adjustment rod 8′ is provided. It comprises an elongated shank 80′ having a worm thread 81′ at its front tip which acts as a worm spindle 45 for the worm drive 46. In a rearward section of the shaft 80′ a fine pitch thread 82′ is provided. Towards its rear end an abutment collar 85′ and finally an elongated crank socket 84′ are provided. For usage, the adjustment rod 8′ is inserted through the hollow guide rod 67′, until its worm thread 81′ at the tip reaches into the second leg 3 of the implant such as to mesh with one of the driven wheels 42 of the worm drive 46. By a clockwise rotation of the adjustment rod 8′ the lifting plate could be raised by virtue of the worm drive 46 of the implant (see FIG. 12b).

The length of shank 80′ is selected such as to have a distance between a rear surface of the abutment collar 85′ to the worm thread 81′ to be shorter, preferably about 3-6 mm, than the distance between the rear face of the annular ring 73′ to the second thread 71′ of the expansion rod 7′. Thereby the rear surface which forms an abutment surface for the projections 608 on the action lever 606 is more forward for the adjustment rod 8′ (as opposed to the expansion rod 7′) such that the handle 601 opens wider. As a result the handle 601 takes its overextended open position, in which the arresting arm 605 with the angled portion of its hybrid tip 607 acts in a wedge-like manner on the interior facing surface of the movable handle 603. Thereby the movable handle 603 is blocked, effectively immobilizing the handgrip. As a result, the projections 608 are fixed in their position and thus form a stable reference for the adjustment rod 8′, by virtue of the—now position fixed—projections 608 abutting against the rear face of the collar 85′. Thereby it is assured that the worm thread 81′ at the tip of the adjusting rod 8′ remains engaged with the driven wheel 42 of the worm drive 46, even in a case where the adjustment rod 8′ is turned counter-clockwise in order to reduce height of the lifting plate if it was overextended.

For actuating the adjustment rod 8′ in order to raise the lifting plate 40, a crank 88′ is provided which can be placed on the receiving socket 84′ of the adjustment rod 8′. By virtue of the crank 88′ a much higher torque can be applied by the surgeon to the adjustment rod and consequently to the worm drive 46, thereby providing a much stronger lifting force. This is an important advantage since spreading of the adjacent vertebrae, as it is effected by the raising lifting plate 40 (see FIG. 13) requires a lot of force due to counter forces induced by ligaments and adjacent tissue (not shown). Owing to the worm drive 46 and its actuation by the crank 88′ this force can be easily and controllably be overcome by the surgeon.

On the fine pitch thread 82′ rides an indicator sled with a marking 89′ which is configured with an inner complementary fine pitch thread engaging the fine pitch thread 82′. The indicator sled with the marking 89′ is guided within the main casing 600 such as to be movable lengthwise but unable to rotate. Thereby the indicator sled 89′ travels lengthwise, namely backwards, as a result of rotating the adjustment rod 8′ clockwise in order to raise the lifting plate 40 of the implant. The indicator sled 89′ co-operates with the 65′ thereby providing a visual reference for the height to which the lifting plate 40 of the implant was already raised.

The adjustment rod 8′ is provided with a needle-like projection at its tip. The needle-like projection acts as a center bearing pin 86′ and is configured such as to be received by a conical recess formed at an interior surface within the second leg 3 of the implant. By virtue of this the tip and the worm thread 81′ are well centered and thus co-operate with the driven wheel 42 in a stable and efficient manner. Further, any friction force that may act on the tip will be concentrated to said needle-like projection, thereby minimizing parasitic torque due to the small arm involved owing to the small width of the needle-like projection. By reducing parasitic torque actuation of the adjustment rod 8′ is facilitated for the surgeon, and further the reduction in friction allows for a more precise adjustment. Such a center bearing pin 86′ may also be provided at the tip of the adjustment rod 8 of the first instrument set at the beginning of the section.

Once the lifting plate 40 has reached its desired height the crank 88′ is to be removed and the adjustment rod 8′ is to be taken out of engagement with the driven wheel 42 and removed from the holding instrument 6. The lifting plate 40 will keep its raised position owing to the self-locking feature of the lifting spindles 41 and the driven wheels 42.

Further, a trial tool 100 is provided in the instrument set. It comprises an elongated body 102 having a trial implant 103 attached as an integral part to a front end. At a rear end a handle 101 is provided. The dimensions and the constitution of the trial implant 103 correspond to that of the implant as described above, except the trial implant lacks the raising lifting plate 40 and its worm drive and further is generally thinner than the implant 3. Thereby insertion into the intervertebral space is facilitated.

The handle 101 comprises a stationary handle 106 and an actuating handle 104 which is coupled to the trial implant 103 by means of a push rod 105. Thereby a backward moving of the actuating handle 104 towards the stationary handle 106 is transferred to the trial implant 103 such as to bring the trial implant 103 out of its flat V-position into an oblique V-position.

In an idle state the trial implant 103 is in flat V-position and thus forms a linear extension of the elongated body 102 of the trial tool (s. FIG. 14b). By backward movement of the actuating handle 104 the trial implant is transferred into its terminal oblique V-position (s. FIG. 14c). This can be reversed by an opposite movement of the actuating handle. By virtue of this tool it can be checked whether there is sufficient space laterally in order to allow the implant to reach its terminal oblique V-position, otherwise the intervertebral space must be reamed further by a reamer (not shown).

1. An intervertebral fusion implant for fusion of two adjacent vertebrae, comprising;
a main body having a first leg, a second leg, and an intermediate articulated joint that is configured as a V-shape adjustable between a flat V-position and an oblique terminal V-position,
a pivoting blade having two ends, a first end being rotatably attached to an end region of the first leg far from the articulated joint,
a guiding trough formed at the second leg, which comprises two lateral side walls and two end-faces, one of the two end-faces being stationary and one being movable with respect to the guiding trough,
wherein the moving end-face is formed by a traveller movable along the guiding trough such as to vary its distance to the stationary end-face, and the traveller is rotatably attached to a second end of the pivoting blade,
wherein an attachment device for engaging a holding instrument is provided at the stationary end-face, and a second attachment device for engaging a pulling organ is provided at the traveller, the traveller providing an aperture for passage of the holding instrument, and further a lifting mechanism is provided, the lifting mechanism comprising a lifting plate adjustable in height between a retracted state and a raised state in which the lifting plate is spaced apart from a top surface of the legs and forms a bearing for an end-plate of an adjacent vertebrae.

2. The intervertebral fusion implant of claim 1, wherein the pivoting blade is solid.

3. The intervertebral fusion implant of claim 1, wherein a plug is provided for the aperture at the traveller.

4. The intervertebral fusion implant of claim 1, further comprising a locking device that is configured for arresting the terminal V-position.

5. The intervertebral fusion implant of claim 4, wherein the locking device comprises a detent.

6. The intervertebral fusion implant of claim 4, wherein the locking device comprises a friction fit between the traveller and the guiding trough.

7. The intervertebral fusion implant of claim 4, wherein the locking device comprises an over-the-centre mechanism for the pivoting blade and the traveller.

8. The intervertebral fusion implant of claim 1, wherein the first and second legs are dimensioned such that they form an isosceles triangle with the pivoting blade.

9. The intervertebral fusion implant of claim 1, wherein the lifting plate in its retracted state is flush with the top cover of the first leg.

10. The intervertebral fusion implant of claim 1, wherein the lifting mechanism comprises a worm gear with worm and worm wheel.

11. The intervertebral fusion implant of claim 10, wherein the worm is configured to be insertable and removable.

12. The intervertebral fusion implant of claim 10, wherein the worm sits at the tip of an actuating instrument for delivery from outside.

13. The intervertebral fusion implant of claim 1, wherein an access hole for driving the lifting mechanism is provided in a wall of the first leg, and the access hole is positioned to be aligned with the aperture of the traveller.

14. The intervertebral fusion implant of claim 1, wherein at least two lifting elements are provided for the lifting plate, the at least two lifting elements being coupled by a wheel drive for synchronous movement.

15. The intervertebral fusion implant of claim 1, wherein a single lifting element is provided for the lifting plate.

16. An instrument set for an intervertebral fusion implant that comprises a main body having a first leg, a second leg, and an intermediate articulated joint that is configured as a V-shape adjustable between a flat V-position and an oblique terminal V-position, a pivoting blade having two ends, a first end being rotatably attached to an end region of the first leg far from the articulated joint, a guiding trough formed at the second leg, which comprises two lateral side walls and two end-faces, one of the two end-faces being stationary and one being movable with respect to the guiding trough, wherein the moving end-face is formed by a traveller movable along the guiding trough such as to vary its distance to the stationary end-face and the traveller is rotatably attached to a second end of the pivoting blade, wherein an attachment device for engaging a holding instrument is provided at the stationary end-face, and a second attachment device for engaging a pulling organ is provided at the traveller, the traveller providing an aperture for passage of the holding instrument, and further a lifting mechanism is provided, the lifting mechanism comprising a lifting plate adjustable in height between a retracted state and a raised state in which the lifting plate is spaced apart from a top surface of the legs and forms a bearing for an end-plate of an adjacent vertebrae, the instrument set comprising:
a holding instrument with an holder device at its front end,
an actuating rod insertable into the holding instruments, the actuating rod comprising an engagement device at a front end for engagement with the second attachment device of the implant at the traveller of the implant, the engagement device being movable along the guiding trough of the implant, and a driving element at its rear end, and
an adjustment rod insertable into the holding instrument, the adjustment rod having a coupling for a lifting mechanism of the implant at its front end.

17. The instrument set of claim 16, wherein a rear end of the actuating rod is configured as a hammerhead.

18. The instrument set of claim 16, wherein the holding instrument comprises:
a guiding tube as an outer part, and
a hollow guide rod as an inner part, wherein the holder device is attached to the guide rod.

19. The instrument set of claim 16, further comprising an axial locking element switchable between a blocked state in which axial movement of the actuating rod relative to the holding instrument is blocked and a free state allowing such axial movement.

20. The instrument set of claim 19, wherein the axial locking element comprises a fixing sleeve that is engaged to the actuating rod.

21. The instrument set of claim 19, further comprising a reference sleeve for the adjustment rod, the reference sleeve being configured for co-operating with the axial locking element such as to arrest the adjustment rod in its axial position.

22. The instrument set of claim 16, wherein a length of the actuating rod is such that, in a fully inserted position, a tip of the actuating rod is located in a region of the traveller of the implant that is attached to the holding instrument.

23. The instrument set of claim 16, wherein the holding instrument is configured as a guiding plier comprising a guiding tube and an actuating handgrip with at least one movable handle having a gripping portion and an action lever portion.

24. The instrument set of claim 23, wherein the action lever portion comprises a coupling connectable to other instruments, and the movable handle is detachable.

25. The instrument set of claim 24, wherein at least one of the actuating rod and the adjusting rod is provided with a connecter for attaching to the coupling.

26. The instrument of set of claim 25, wherein the coupling and the connecter are configured for a form-fit.

27. The instrument set of claim 25, wherein the coupling and the connecter are releasable in at least two different positions of the handgrip that include a relaxed position and a squeezed position.

28. The instrument set of claim 25, wherein an arresting device for the handgrip is provided that is configured to arrest the movable handle.

29. The instrument set of claim 25, wherein the connecter on the adjustment rod is offset forward compared to the connector on the actuating rod.

30. The instrument set of claim 28, wherein the arresting device is configured to be activated upon engagement of the coupling into the connecter of the adjustment rod.

31. The instrument set of claim 16, wherein a crank is provided to be releasably connected to a rear end of the adjustment rod.

32. The instrument set of claim 16, wherein a check mark device is provided for at least one of the actuating rod and the adjustment rod, the check mark device comprising a movable indicator on the at least one of the actuating rod and the adjustment rod and a scale configured to show a position of the implant.

33. The instrument set of claim 16, wherein an observation window is provided along a side of the holding instruments, the observation window providing visual access to at least one of the actuating rod and the adjustment rod inserted in the holding instrument.

34. The instrument set of claim 16, wherein a front surface of the holding instrument is provided with a planar contact region for abutting to the implant.

35. The instrument set of claims 16, wherein the holding instrument comprises fixation projections on a front face, the fixation projections are configured to co-operate in a form-fit manner with the implant.

36. The instrument set of claim 16, wherein a center bearing pin is provided at a tip of the adjustment rod.

37. The instrument set of claim 16, further comprising a trial tool having a trial implant at a front end and an actuating handle at a rear end, the trial implant being configured to match the configuration of the implant.

38. The instrument set of 37, wherein the trial tool comprises at least one of a depth scale and/a position indicator scale configured for indicating a depth of insertion or an expanding state, respectively, of the V-position of the trail implant.

39. The intervertebral fusion implant of claim 1, wherein the first and second legs are dimensioned such that they form a right triangle with the pivoting blade in the terminal V-position.

40. The intervertebral fusion implant of claim 10, wherein the worm wheel is directly attached to a lifting element of the lifting plate.

41. The intervertebral fusion implant of claim 15, wherein the single lifting element interacts with a rotation preventer.

42. The instrument set of claim 16, wherein the driving element is a handle.

43. The instrument set of claim 25, wherein the connecter is configured for transmission of axial forces.

44. The instrument set of claim 25, wherein the coupling and the connecter are configured for a form-fit by a pair of projections in opposing relation and a matching recess.

45. The instrument set of claim 35, wherein the fixation projections comprise a tongue projecting outward from the front face of the holding instrument, the tongue being configured to interact with a complementary shaped notch at the second leg of the implant.

46. The instrument set of claim 16, further comprising a trial tool having a trial implant at a front end and an actuating handle at a rear end, the trial implant being configured to match the configuration of the implant without the lifting mechanism.
